(19) United States
(12) Patent Application Publication
(10) Pub. No.: US 2021/0369459 A1
(43) Pub. Date: Dec. 2, 2021

(54) VENOUS VALVE WITH ENHANCED FLOW PROPERTIES

(71) Applicant: Innovein, Inc., Rohnert Park, CA (US)

(72) Inventors: Dale L. Ehnes, Forestville, CA (US); Teresa Woodson, Windsor, CA (US); Christopher Staudenmayer, Santa Rosa, CA (US); Austin Walker, Pacifica, CA (US); Thomas Kramer, San Carlos, CA (US); Albert K. Chin, Palo Alto, CA (US); Mark Geusen, Santa Rosa, CA (US)

(21) Appl. No.: 17/336,429

(22) Filed: Jun. 2, 2021

Related U.S. Application Data
(60) Provisional application No. 63/033,312, filed on Jun. 2, 2020.

(51) Int. Cl. A61F 2/24

(52) U.S. Cl. CPC A61F 2/2475; A61F 2230/0076; A61F 2230/0071; A61F 2/2424

(57) ABSTRACT

A prosthetic venous valve includes an expanding anchoring frame, a valve seat at the middle portion of the anchoring frame, a ball disposed within the lumen of the anchoring frame and having an outer diameter, and least one ball retention tether coupled with the ball and the anchoring frame. The ball retention tether includes at least one elastic component or material. The anchoring frame has an upstream end, a downstream end, a middle portion and a lumen extending through the anchoring frame from the upstream end to the downstream end. The ball moves between an open position, in which the ball is located apart from the valve seat, and a closed position, in which the ball is located in contact with or near the valve seat to reduce or prevent backflow of blood through the prosthetic venous valve.

### CROSS-REFERENCE TO RELATED APPLICATIONS

This application claims the benefit of U.S. Provisional Patent Application No. 63/033,312, filed Jun. 2, 2020, entitled, “VENOUS VALVE WITH ENHANCED FLOW PROPERTIES.” The disclosure of this priority application is hereby incorporated by reference in its entirety into the present application.

### TECHNICAL FIELD

This application relates generally to the field of medical devices. More specifically, the application relates to prosthetic valve implant devices, systems and methods for implantation within the vasculature.

### BACKGROUND

Veins in the human body are weak-walled blood vessels that carry blood under low pressures back to the heart from the extremities. To help move the blood toward the heart, most frequently against the force of gravity, veins have one-way valves, which open in the direction of forward-moving blood flow and close to prevent backflow of blood. When these valves become compromised, the veins cannot function properly. Venous disease, due to incompetent venous valves, is a prevalent clinical problem. In the U.S., 20 million patients demonstrate chronic venous insufficiency, with swelling, pain, and/or ulceration of the affected extremity. An additional 74 million patients exhibit the dilation and deformity of varicose veins.

Various approaches have been advanced for addressing the clinical problem of poorly functioning venous valves. Mauch et al. (U.S. Pat. No. 7,955,346) teach a percutaneous method for creating venous valves from native vein tissue. Laufer et al. (U.S. Pat. No. 5,810,847) describes catheter placement of a clip appliance onto the cusp of a valve to restore the function of incompetent lower extremity venous valves. Multiple designs for implantable venous valves have also been described. These designs involve implantable prosthetic valves that mimic the patient's natural (autologous) valves; that is, the implants use pliable leaflet or flap valves to restore unidirectional venous flow. Examples of such implantable venous valves are described by Acosta et al. (U.S. Pat. No. 8,246,676), Shaolian et al. (U.S. Pat. No. 6,299,637), and Thompson (U.S. Pat. No. 8,377,115), for example.

In order to mimic native human peripheral venous valves, leaflet or flap valves are formed of extremely thin membrane material, to allow the valve to open properly for return flow to occur in the low pressure venous system, while still providing proper sealing and avoiding valvular insufficiency. Prosthetic membrane or flap valves are prone to failure, due to tearing from repeated opening and closing of the leaflets, permanent closure due to thrombosis and cell adhesion to the prosthetic leaflets, or leaflet inversion and incompetence over time. Currently available replacement venous valves, whether artificial or transplanted tissue valves, also often cause problems with thrombosis or clotting during long term implantation.

Therefore, it would be advantageous to have improved implantable venous valves, which would be designed to address these challenges. It would desirable, for example, to have a prosthetic venous valve that prevents and/or accommodates for the occurrence of thrombosis or cell adhesion to the valve components during long-term valve implantation. Ideally, the improved prosthetic valve would be relatively easy to implant and would address at least some of the challenges of currently available valve implants discussed above.

### BRIEF SUMMARY

The embodiments described herein are directed to implantable, prosthetic vascular valve devices, systems and methods for their use. Typically, the vascular valve implants described herein are used in veins, to replace or do the work of faulty or nonexistent venous valves. However, the implants may be used in arteries or other structures in the human body, such as heart valves or other body lumens that might benefit from a prosthetic valve. Thus, the description herein of venous valve implants may also be applied to arteries and other anatomical structures.

The prosthetic venous valve assembly described in this application (sometimes referred to simply as a “venous valve” or “venous valve implant”) generally includes an anchoring portion and a mobile valve component. The anchoring component (typically referred to herein as an “anchoring frame” or “anchoring member”) in many embodiments is a self-expanding frame, although alternatively it may be expandable by other means, such as balloon expandable. The anchoring frame may be bare, partially covered or completely covered in a coating or graft material. In all of these embodiments, the anchoring frame forms a lumen from end to end. The lumen of the anchoring portion typically includes a narrowed portion (also referred to herein as the “valve seat” or “waist”) and in some cases is shaped like an hourglass.

The mobile valve component is typically referred to herein as a “ball,” although in some embodiments it may not be shaped like a ball. The ball may be spherical in shape or may have other suitable shapes, such as ovoid, football-shaped, lemon-shaped, cone-shaped or the like. The venous valve implant also includes some form of tethering or retention member (or members) for attaching the ball to the anchoring portion or otherwise preventing the ball from exiting the anchoring portion. When implanted in a vein, the mobile valve component moves away from the waist of the valve when blood flows through the valve in a forward (returning to the heart) direction, thus opening the valve and allowing forward flow of blood. The mobile valve component then drops back into contact (or at least proximity) with the narrow waist portion of the valve to close the valve and minimize or prevent backflow of blood (or “regurgitation”) through the valve. A number of different embodiments, variations and features of this implantable venous valve prosthetic device are described herein.

The mechanical nature of the prosthetic ball valve confers durability to the implant, thereby removing the modes of valve failure observed in prosthetic leaflet valves. With a ball valve, no leaflets are present that may thicken, tear or prolapse. In the prosthetic venous ball valve, the failure mode is limited to clot formation (or “thrombosis”). Therefore, it is desirable to incorporate a ball design and retention system that minimize the potential for thrombus formation within the structure of the valve assembly. Thrombus formation is dependent on the characteristics of blood flow (hemodynamics) through the valve, where flow is maintained inside the lumen formed by the implant's anchoring portion and over the mobile ball component. The orifice size of the valve seat should be maximized to avoid high fluid resistance to forward venous blood flow. Maximizing the valve seat orifice size and the ball size (which is oversized relative to the orifice to contact the valve seat), however, creates a ball with a large outer profile that is difficult to insert into position in a patient's vein. If a venous valve implant contains a self-expanding frame (or even a balloon expandable frame), the frame may reside in a compressed configuration for delivery into the vein. If a rigid ball is used in the valve assembly, its diameter becomes the limiting factor in the ability for the valve assembly to compress into a decreased delivery profile. Thus, a compressible ball is included in many embodiments described herein, to minimize the delivery system profile of the prosthetic venous valve implant. In an alternative embodiment, the ball and the orifice have similar diameters, and the ball resides fully within the orifice (thereby acting like a “plug”) when the ball is in the closed position. This configuration may allow for a smaller ball and/or a larger valve seat orifice, either or both of which may help reduce forward blood flow resistance.

Implants constructed with foreign materials can lead to clot formation in the bloodstream. The characteristics of blood flowing through the valve may also contribute to thrombus formation. When the valve opens due to forward blood flow, the ball moves out of contact with the valve seat due to forward pressure to its “open” position, as allowed by the retention constraints. The valve closes due to retrograde flow and elastic spring force (in some embodiments), creating a pressure reversal on the ball and the ball moves back into contact with the valve seat. During the excursions of the ball in and out of contact with the valve seat, the flow conditions are in a transitory state for a short duration of time. When the ball is in the open position, however, the path of blood between the ball and the inner surface of the frame should ideally be uniform (i.e., laminar flow, no stagnant areas, etc.). Maintaining a uniform flow path area between the ball and the inner surface of the frame avoids changes in blood flow velocity that lead to eddy currents and areas of static blood flow at different locations within the valve assembly. Areas of stasis in the valve contribute to clot formation or thrombosis. Additionally, venous blood flow is susceptible to thrombogenesis when wall shear rates are too low or too high. Optimally, wall shear rates of venous implants may be designed to be within a desired range, to mitigate thrombus formation. Further, if implanted devices are too restrictive with respect to forward flow back to the heart, alternative flow paths around the device in the target vessel or in nearby vessels may develop, which may lead to thrombogenesis due to reduced flow and stagnation in the implant. It is therefore believed that a prosthetic venous valve assembly should have low resistance to forward flow. This is achieved by designing the prosthetic venous valve assembly to have a low pressure drop for a given flow rate and low stiffness for embodiments with an elastic retention system. The venous valve prosthetic devices described herein include features and design characteristics that advantageously address many if not all of these issues of flows, shear, thromobogenesis and the like.

Embodiments of a venous valve implant are described in this application for improving the fluid flow within the vascular valve assembly. In most if not all embodiments, the ball of the implant (or “the valve component”) is collapsible/expandable (or “non-rigid”). It is desirable to modulate the axial position of the ball as a function of flow rate, and the embodiments herein include features to address this goal. For low flow rates, the ball may be elastically pulled to the closed position (or nearly closed) at the waist with low force, to enable valve motion (i.e., flutter). This position at low flow rates prevents the ball from resting on the frame (valve stasis) further downstream, which may cause occlusion of the implantable valve. Conversely, in order to accommodate relatively large flow rates (e.g., during exercising), the ball moves to a more open, less restrictive position to mitigate high wall shear rates that may lead to thrombus (clot) formation. Thus, embodiments described herein provided a variable position valve according to variable input flow rates, thereby helping reduce thrombus formation at both low and high flow rates.

Some embodiments also provide for a more centered ball within the valve assembly, in order to prevent the ball from resting on the frame of the assembly (valve stasis), which can cause clot formation and occlusion of the valve system. Such embodiments may incorporate a spring or elastic component or portion. In some embodiments, the spring/elastic component may work in conjunction with one or more inelastic tethers.

In one aspect of the present disclosure, a prosthetic venous valve may include: a self-expanding anchoring frame having a proximal end, a distal end, and a lumen extending through the anchoring frame from the proximal end to the distal end; a valve seat formed in or attached to the anchoring frame, the valve seat having an inner diameter; an expandable ball disposed within the lumen of the anchoring frame and having an outer diameter; and a ball retention tether attached to the expandable ball and to the anchoring frame. The expandable ball expands from a compressed configuration for delivery into the vein through a delivery catheter to an expanded configuration outside the delivery catheter, and the expandable ball in the expanded configuration moves between an open position, in which the expandable ball is located apart from the valve seat, to allow forward flow of blood through the implant, and a closed position, in which the expandable ball contacts the valve seat to reduce or prevent backflow of blood through the implant. The inner diameter of the valve seat, the outer diameter of the expandable ball, and/or a length/stiffness of the ball retention system are all configured to provide for a desired flow pattern of blood through the prosthetic venous valve.

In some embodiments, the anchoring member may be a stent that extends from a proximal end to a distal end of the implant and forms a lumen from the proximal end to the distal end. The anchoring frame may include a cylindrical proximal portion at the proximal end, a cylindrical distal portion at the distal end, an inwardly angled inlet portion between the cylindrical proximal portion and a middle of the anchoring frame, and an inwardly angled outlet portion between the cylindrical distal portion and the middle of the anchoring frame.

In some embodiments, the valve further includes a membrane disposed over at least part of the anchoring frame, where the membrane is made of polymers, hyaluronic acid, heparin and/or anticoagulant agents. In some embodiments, the anchoring frame includes multiple outward facing protrusions on the proximal end and/or the distal end. In some embodiments, the anchoring frame has an hourglass shape, and the valve seat is a narrow portion of the anchoring frame, between the proximal end and the distal end. In alternative embodiments, the anchoring frame has a bowtie shape, where the anchoring frame narrows as it extends toward a middle portion from the proximal end and the distal end and then has a larger diameter at the middle portion than at immediately adjacent regions of the anchoring frame, next to the middle portion. In some embodiments, the inner diameter of the valve seat is slightly larger than the largest outer diameter of the ball, thus allowing the ball to pass into the valve seat and act as a plug. For example, the inner diameter of the valve seat may be up to 0.5 millimeters larger than the largest outer diameter of the ball.

In various embodiments, the ball may have any of a number of suitable shapes, such as but not limited to spherical, prolate spheroid, ellipsoid, ovoid, egg shaped or asymmetrical. In one embodiment, for example, the ball has a prolate spheroid shape and includes an expandable lattice structure (or “frame”) and a membrane covering the expandable lattice. As the term “frame” is used often throughout this disclosure to describe the anchoring frame of the venous valve prosthetic device, the term “lattice” or “lattice structure” will be typically be used to describe the frame portion of a ball, when the ball has a frame/lattice and a covering or coating.

In some embodiments, the tether has a V-shape with a first end attached to the anchoring frame, a middle portion attached to the ball, and a second end attached to the anchoring frame. Alternatively, the V-shape may be formed by using two tethers, each of which is attached at one end to the anchoring frame and at an opposite end to the ball. In some embodiments, the tether is attached to eyelets on the ball and the anchoring frame. In other embodiments, the tether is attached to the covering on the ball and the anchoring frame. In some embodiments, the V-shape forms an obtuse angle, while in other embodiments, the V-shape forms an acute angle. In some embodiments, the tether is elastic or incorporates a spring element to impart elasticity. In some embodiments, the ball further includes a loop for connecting the tether to the ball.

In another aspect of the present disclosure, a method for treating a vein in a human subject first involves advancing a delivery catheter containing a prosthetic venous valve into the vein. The prosthetic venous valve may include any of the aspects and features described above. The method further involves delivering the prosthetic venous valve into the vein and removing the delivery catheter from the vein.

In some embodiments, the expandable ball is a solid, compressible foam ball. Such embodiments may optionally further include at least one weight embedded within the ball. Alternatively, the expandable ball may include an elastic shell and a filler substance inside the elastic shell. For example, the filler substance may be air, a gel or a fluid. Some embodiments include at least one weight inside the elastic shell. Optionally, the filler substance may be a curable substance that hardens when cured. In some embodiments, the filler substance is a spiral-cut, elastic, hollow sphere. In some embodiments, the expandable ball includes an aperture through which the ball retention tether is passed. In some embodiments, the expandable ball is bare with no covering. In some embodiments, the expandable ball has a density of less than 2.5 grams per square centimeter. The ball retention tether is attached to the valve seat, and the tether and the valve seat form a filling lumen, and the valve seat is accessible through a filling port to pass a filler substance through the valve seat and the tether to fill the expandable ball. In some embodiments, the expandable ball has a density of no greater than 1.06 grams per square centimeter, and the tether is elastic, to pull the ball toward the valve seat to prevent backflow of blood through the implant or to modulate the valve position in forward flow. In various embodiments, the expandable ball may be made of a material such as but not limited to thermoplastic polyurethane, elastomeric thermoplastic polyurethane, PVC, Polyethylene, polycarbonate, PEEK, ultem, PEI, polypropylene, polysulfone, FEP, PTFE, ePTFE, Nitinol, coated hollow heavy metal or combinations thereof.

In another aspect of the present disclosure, a prosthetic venous valve includes an expanding anchoring frame having an upstream end, a downstream end, a middle portion and a lumen extending through the anchoring frame from the upstream end to the downstream end. The prosthetic venous valve also includes: a valve seat including a portion of the middle portion of the anchoring frame; a ball disposed within the lumen of the anchoring frame, where the ball moves between an open position, in which the ball is located apart from the valve seat, and a closed position, in which the ball is located in contact with or near the valve seat to reduce or prevent backflow of blood through the prosthetic venous valve; and at least one ball retention tether coupled with the ball and the anchoring frame, wherein the at least one ball retention tether comprises at least one elastic component or material.

In some embodiments, the prosthetic venous valve may further include a membrane disposed over at least part of the anchoring frame. Such a membrane may be made of one or more substances, such as but not limited to polymers, hyaluronic acid, heparin and/or anticoagulant agents. In one embodiment, the anchoring frame has an asymmetric shape, and a downstream portion of the anchoring frame is longer than an upstream portion of the anchoring frame. In some embodiments, the valve seat is a tapered portion of the middle portion of the anchoring frame. In various embodiments, the anchoring frame may have a shape such as but not limited to an hourglass shape, a bowtie shape and an asymmetrical shape. In some embodiments, the inner diameter of the valve seat is larger than a largest outer diameter of the ball by 0.5 millimeters or less, thus allowing the ball to pass into the valve seat and act as a plug.

In various embodiments, the ball may have any of a number of suitable shapes, such as but not limited to spherical, prolate spheroid, ellipsoid, ovoid, egg shaped, lemon shaped and asymmetrical. In some embodiments, the ball is expandable. An expandable ball may include an expandable lattice a membrane covering the expandable lattice. In some embodiments, the membrane includes at least one aperture for allowing blood to pass into an interior of the ball.

In some embodiments, the ball retention tether has a V-shape with a first end attached to the anchoring frame, a middle portion attached to the ball, and a second end attached to the anchoring frame. In some embodiments, the ball retention tether includes a main tether member, attached at one end to the ball and at an opposite end to the anchoring frame, and the elastic component is a spring disposed over a portion of the main tether member and attached at one end to the anchoring frame. In some embodiments, the main tether member may be attached to eyelets on the ball and on the anchoring frame. In some embodiments, the main tether member is attached to a covering on the ball and to a covering on the anchoring frame. In alternative embodiments, the ball retention tether may include a first tether attached to the anchoring frame and a second tether attached to the ball, and the elastic member is a spring connecting the first tether to the second tether. In some embodiments, the ball retention tether is made of an elastic material.

In alternative embodiments, the ball retention tether may include an upstream tether attaching the ball to an upstream portion of the anchoring member and a downstream tether attaching the ball to a downstream portion of the anchoring member. Alternatively, the ball retention tether may include multiple upstream expandable fingers extending from an upstream end of the ball and multiple downstream expandable fingers extending from a downstream end of the ball. In other embodiments, the ball retention tether includes a first tether attached to the anchoring frame and a second tether attached to the ball, and the elastic member is a hinge connecting the first tether to the second tether. In some embodiments, the ball further includes a loop for connecting the ball retention tether to the ball.

In another aspect of the invention, a prosthetic venous valve includes: an expanding tubular anchoring frame extending from a first end to a second end of the venous valve prosthesis, forming a lumen; a valve seat formed by or attached to the anchoring frame; a ball in the lumen of the anchoring frame; and at least one ball retention tether attached to the ball and the anchoring frame, where the at least one ball retention tether comprises at least one elastic component or material.

In some embodiments, the elastic component is a spring disposed over a main tether member and attached to the anchoring frame. In some embodiments, the ball and the ball retention tether are configured so that the ball moves between an open position, in which the ball is located downstream of the valve seat, and a closed position, in which the ball is located closer to the valve seat or contacts the valve seat, to reduce or prevent backflow of blood. In some embodiments, the ball is expandable from a compressed configuration for delivery into the vein through a catheter to an expanded configuration outside the catheter.

These and other aspects and embodiments are described in further detail below, in reference to the attached drawing figures.

### BRIEF DESCRIPTION OF THE DRAWINGS

FIG. 1 is a side view of a prosthetic venous valve including a ball and a tether, according to one embodiment;

FIG. 2 is a perspective view of a compressible prolate spheroid ball of a prosthetic venous valve, with a cover and loop attachment site, according to one embodiment;

FIGS. 3A and 3B are diagrammatic views illustrating the principles of fluid mechanics that govern blood flow through a venous valve;

FIG. 4A is a side view of an hourglass shaped anchoring member and spherical ball of a prosthetic venous valve, according to one embodiment;

FIG. 4B is a flow diagram illustrating a velocity profile of blood flow through the venous valve of FIG. 4a when the valve is in the open position;

FIG. 5A is a side view of a bowtie shaped anchoring member and prolate spheroid ball of a prosthetic venous valve, according to an alternative embodiment;

FIG. 5B is a flow diagram illustrating a velocity profile of blood flow through the venous valve of FIG. 5a when the valve is in the open position;

FIGS. 6A-6D are diagrammatic views illustrating fluid flow velocity profiles for alternative configurations of a venous valve prosthesis; FIG. 6A shows an hourglass frame and spherical ball; FIG. 6B shows an hourglass frame and egg shaped ball; FIG. 6C shows an hourglass frame and spherical ball; and FIG. 6D shows an hourglass frame and prolate spheroid ball;

FIGS. 7A and 7B are side views of two alternative embodiments of a prosthetic venous valve with two V-shaped tethers; FIG. 7A shows a shorter tether that forms an obtuse angle; FIG. 7B shows a longer tether that forms an acute angle;

FIG. 8A is a side view of a prosthetic venous valve with direct attachments of the V-shaped tether to the anchoring member and the ball, according to one embodiment;

FIGS. 8B-8D are side, close-up and front views, respectively, of a prosthetic venous valve with eyelet attachments of the V-shaped tether to the anchoring member and the ball, according to an alternative embodiment;

FIGS. 9A and 9B are side views of a prosthetic venous valve in the open and closed positions, respectively, with a ball having an outer diameter just slightly smaller than an inner diameter of the valve orifice, according to one embodiment;

FIG. 10 is a side, cross-sectional view of an expandable anchoring frame and an expandable ball of a venous valve prosthetic device, according to one embodiment;

FIG. 11 is a side, flattened view, and a close-up view, of the anchoring frame of the device of FIG. 10;

FIG. 12 is a side, cross-sectional view of a venous valve prosthetic device in a closed position, with the anchoring frame and expandable ball of FIG. 10 and also with a tether with a spring component, according to one embodiment;

FIG. 13 shows the venous valve prosthetic device of FIG. 12 in an open position;

FIG. 14 is a side view of an embodiment of a ball retention tether that includes two portions of overlapping tether connected by a spring;

FIG. 15 is a side view of an embodiment of a ball retention tether that includes a spring disposed over a portion of the tether that extends beyond the ball;

FIG. 16 is a side view of an embodiment of a ball retention tether that includes two portions of non-overlapping tether connected by a spring;

FIG. 17 is a side view of an embodiment of a ball retention tether made of an elastic material;

FIGS. 18 and 19 are side, cross-sectional views of another embodiment of a venous valve prosthetic device, in which the ball flips into (FIG. 18) and out of (FIG. 19) a tether extension to open and close the valve;

FIG. 20 is a side, cross-sectional view of a venous valve prosthetic device with two tethers and two springs, according to an alternative embodiment;

FIG. 21 is a side, cross-sectional view of a venous valve prosthetic device with two V-shaped tethers and two springs, according to another alternative embodiment;

FIG. 22 is a side, cross-sectional view of a venous valve prosthetic device with two tethers and two flexible beams, according to another alternative embodiment;

FIG. 23 is a side, cross-sectional view of a venous valve prosthetic device with one tether having two portions connected by a hinge, according to another alternative embodiment;

FIGS. 24 and 25 are side, cross-sectional views of a venous valve prosthetic device with a spring tether, in an expanded configuration as inside of a vein (FIG. 24) and a collapsed configuration inside a delivery catheter (FIG. 25), according to another alternative embodiment;

FIG. 26 is a side, cross-sectional view of a venous valve prosthetic device with a ball connected to a spring, according to another alternative embodiment;

FIG. 27 is a side, cross-sectional view of a venous valve prosthetic device with two overlapping tether portions connected by a spring, according to another alternative embodiment;

FIG. 28 is a side, cross-sectional view of a venous valve prosthetic device with a ball connected to a spring, both of which are downstream of the ball of the device, according to another alternative embodiment;

FIG. 29 is a side, cross-sectional view of a venous valve prosthetic device with a two portions of tether connected by a hinge, with a spring disposed over a distal section of the tether, according to another alternative embodiment;

FIG. 30 is a side view of an expandable ball of a venous valve prosthetic device with a fin or hemofoil extending from it, illustrated during manufacturing before the fin has been cut, according to one embodiment;

FIGS. 31 and 32 are side and posterior views, respectively, of the ball with fin of FIG. 30, after the fin has been cut to a shorter length during manufacturing;

FIGS. 33 and 34 are side views of a ball of a venous valve prosthetic device with fins or hemofoils on opposite sides, according to another alternative embodiment;

FIG. 35 is a side view of a ball of a venous valve prosthetic device with fins or hemofoils on opposite sides and tethers attaching the fins to the anchoring frame, according to another alternative embodiment;

FIGS. 36A and 36B are side and posterior views, respectively, of an expandable ball with a plug shape, according to an alternative embodiment;

FIG. 37 is a side view of an expandable ball with a figure-eight shape and a fin or hemofoil, according to another alternative embodiment;

FIGS. 38 and 39 are side and posterior views, respectively, of another expandable ball with a figure-eight shape and a fin or hemofoil, according to another alternative embodiment;

FIGS. 40 and 41 are side views with different rotational orientations of an expandable ball with fins on either side, according to another alternative embodiment;

FIGS. 42 and 43 are side and perspective views, respectively, of an expandable ball with a fin, where both are made of a braided material, according to another alternative embodiment;

FIG. 44 is a side view of an expandable ball with a fin made of multiple pieces of material, such as ePTFE, according to another embodiment;

FIGS. 45 and 46 are side and perspective views, respectively, of an expandable ball with a fin configured similar to a box kite, according to another alternative embodiment;

FIG. 47 is a side view of an expandable ball with fingers extending from opposite sides of the ball, where the ball and fingers are covered in a material such as ePTFE, according to another embodiment;

FIG. 48 shows the ball with fingers of FIG. 47 within a manmade vein test structure; and

FIG. 49 show shows the ball with fingers of FIG. 47 without the covering.

### DETAILED DESCRIPTION

In this disclosure, the term “proximal” will be used synonymously with “upstream,” and the term “distal will be used synonymously with “downstream.” “Upstream,” in this disclosure, means farther away from the heart, in other words upstream when blood is flowing toward the heart. “Downstream,” in this disclosure, means closer to the heart, in other words downstream when blood is flowing toward the heart. Although in a vein blood sometimes flows in a retrograde direction (i.e., away from the heart, for example between heartbeats), the terms upstream and downstream in this disclosure refer to blood flow in the direction toward the heart. Similarly, “forward flow” means blood flow in a direction toward the heart, and “retrograde flow” or “backward flow” means blood flow in a direction away from the heart. “Horizontal flow” or “in a horizontal direction,” refers to blood flow in a direction straight through the blood vessel and/or the valve implant device. This definition is used regardless of the orientation in space of the blood vessel itself.

The prosthetic valve assembly described herein is commonly referred to in this disclosure as a “venous valve prosthetic device” or “simply venous valve prosthesis.” As mentioned above, any given embodiment may be used (or adapted for use) in arteries, heart valve, or other body lumens. Thus, the scope of this disclosure is not limited to use of device in veins.

A prosthetic venous valve described herein is composed of A) an expandable tubular anchoring frame extending from a first end to a second end. In some embodiments the tubular frame may form a lumen to direct blood flow. It may also define the terminal ends of the venous valve prosthesis. In some embodiments part or all of the frame may be coated or covered with materials such as anti-clotting agents (e.g. heparin) to mitigate clot formation, or polymers (e.g. ePTFE) to direct the flow of blood. In some embodiments the frame may be shaped as an hourglass or bowtie. In some embodiments the contour and area of the frame may be modified to minimize shear or turbulence within the implant. It may contain a lower-radial force section or sections that allows the implant to taper back down to nominal vessel size. It may contain one or more active anchoring mechanisms such as barbs to prevent migration. B) a valve seat formed by or attached to the anchoring frame. In some embodiments the valve seat may be formed by the frame itself or a material coupled with the frame. Alternatively the design may encourage vessel attachment and ingrowth into the device, where in some embodiments the tissue may form a part of or the entirety of the valve seat. C) a ball in the lumen of the anchoring frame. The ball may be collapsible and expandable or non-collapsible and non-expandable. It may be formed of and/or coated by various materials such as polymers, metals, anti-clotting agents (e.g. heparin). It may be formed of different shapes such as a prolate spheroid, ellipsoid, ovoid, etc. It may be hollow or filled with another substance or substances (e.g. air, blood, saline). In one embodiment, it is formed of a self-expanding metal (e.g. Nitinol) covered in a polymer (e.g. ePTFE). D) at least one retention tether coupled with the ball and the anchoring frame or vessel wall, wherein the at least one ball retention tether comprises at least one elastic component or material. In some embodiments the tether itself may be formed of an elastic material such as Neoprene, silicone, rubber elastomers, etc. In some embodiments, the tether may be couple with, or pass through, an elastic material such as a Nitinol coil. All tether and/or coupled elastic materials may be further coated in other materials (e.g. ePTFE, heparin, etc.), which may improve hemocompatibility.

Additional venous valve prosthetic devices described herein include an expandable anchoring frame (self-expandable or balloon expandable, for example), which in many embodiments is formed as a metal, lattice-type structure, similar to that of a stent.). In some embodiments, the upstream and downstream ends of the anchoring frame are wider than the middle section that forms a valve seat. A ball resides downstream of the valve seat, sometimes but not always between the downstream end of the frame and the middle section, where its retrograde movement is restricted by the valve seat and/or one or more tethers (in some embodiments). A ball retention member (e.g., tether, multiple tethers, a spring tether system, etc.) is attached to the ball and to the anchoring frame and defines the open position of the ball during forward flow. The anchoring frame may be completely bare metal or may optionally be coated or covered on one or both sides (outer surface and/or inner surface) by a polymer membrane. Any coating or covering may extend the full length of the anchoring member or may cover only part of the anchoring member, according to various embodiments. The shape of the self-expanding anchoring frame structure may be configured such that with the ball in the open position, the flow area between the surface of the ball and the inner surface of the frame remains a constant value. This constant flow path relationship between the respective surfaces of the ball and the expanding frame may be achieved by configuring the frame such that its inner cross-sectional area transitions in proportion to the radius squared, starting from the narrowed middle portion of the frame towards the largest portion of the valve in the open position. By maintaining a constant area flow path around the ball, favorable blood flow characteristics, including laminar flow and an absence of eddy currents, are provided. Reducing or eliminating eddy currents and regions of stagnant flow in the valve helps to reduce or eliminate the risk of thrombus formation. In other embodiments, the prosthetic venous valve may have an increasing area between the surface of the ball and the inner surface of the covered frame, to slow the flow velocity (or vice versa). Further, implantable valve embodiments may employ a “constant gap” design where the distance (i.e., “gap”) between the surface of the ball and the inner surface of the covered frame is constant to reduce fluid wall shear rates.

The configuration of the tether (or tethers) also factors into the ability of the prosthetic ball valve to resist thrombus formation. A single tether strand may result in an offset ball in the open valve position that rests against the side of the valve, thereby creating a stagnant area of flow that induces thrombus formation. A V-shaped tether with two frame connection points and one ball connection point may center the ball for improved long-term valve patency. A single tether strand incorporating elasticity may preferably modulate the position of the valve based on flow rate thereby preventing stasis at lower flow rates and reducing high shear conditions at higher flow rates.

A prosthetic valve implant may be configured to present a minimal delivery profile by consisting of a super-elastic metal lattice structure frame with a ball valve centered within the frame, and such ball valve comprised of a self-expanding super-elastic lattice shaped as a spherical or prolate spheroid form, or one of many other possible shapes, which is then covered (or otherwise encapsulated) with a polymer membrane. The polymer used to cover the ball may be comprised of expanded polytetrafluoroethylene (ePTFE), polytetrafluoroethylene (PTFE), polyurethane, or a suitable combination such as polyurethane inside the valve lattice and PTFE outside the valve lattice heat bonded to encapsulate the valve lattice and form a membrane that directs blood flow through the lumen and around the ball in vivo. The frame may be fully covered (abluminally, adluminally or fully encapsulated), partly covered by length (also by the aforementioned embodiments), or left bare. In embodiments where the anchoring frame is bare metal (or partially bare metal), the native vein or other blood vessel in which the device is implanted will tend to grow into or through the openings in the lattice of the anchoring frame. In some embodiments, the native tissue may form the valve seat of the device after implantation (or part of the valve seat). The native vein then acts as part of the as the anchoring frame's luminal flow surface (or “inner surface” or “inner wall”). In fact, in some embodiments the anchoring frame is designed to encourage vessel wall ingrowth to used the vessel wall tissue as part of the device itself. The ball may be positioned within the self-expanding frame by a polymer tether attached to the ball and a location (or plurality of locations) on the frame. The polymer tether may be a monofilament formed of ePTFE, PTFE, Nylon, polypropylene, or other material. The tether may also be formed of multiple strands that attach to one or more points on the frame, and one or more points on the covered super-elastic ball. Further, the tether may be elastic or may pass through (or otherwise be affixed to) a spring element to impart elasticity and form a spring-tether system for ball retention.

The self-expanding ball and the self-expanding frame are preferably configured to optimize the characteristics of blood flow through the valve implant. Blood flow through a tubular structure such as the venous valve is governed by the Hagen-Poiseuille Law for laminar fluid flow that states that axial fluid flow velocity assumes a parabolic shape, with the flow velocity equal to zero at the inner wall of the tube and increasing in proportion to the radius squared. The velocity of incompressible blood flowing between the ball and the frame varies linearly according to the cross-sectional area of the flow path, following the formula A1v1=A2v2, where A1 is the cross-sectional area of a tube at point 1, v1 is the fluid flow velocity at point 1, A2 is the cross-sectional area of the tube at point 2, and v2 is the fluid flow velocity at point 2. If the cross-sectional area of fluid flow path decreases, the fluid flow velocity increases accordingly in a linear fashion. Therefore, by maintaining a constant cross-sectional fluid flow path between the ball and the frame wall, blood flow velocity remains uniform. Blood flow velocity changes may create eddy currents, areas of stasis (or reduced flow), or regions of high wall shear rates that may initiate clot formation leading to valve failure. In some embodiments, the venous valve prosthesis described herein includes a constant cross-sectional fluid flow path.

Similarly, the position of the ball during opening and closing of the valve is related to the propensity for post-implant clot formation. If the ball is not centered within the valve, thrombus may preferentially form in a low flow contact region between the ball and the frame. A ball retention system is proposed that utilizes a V-shaped monofilament to attach the ball to the frame. A longer distance between the ball and the frame attachment sites has been demonstrated on bench flow model studies to be superior to a shorter tether distance for higher flow rates. A shorter distance between the ball and the frame attachment sites has been demonstrated on bench flow model studies to be superior to a longer tether distance for lower flow rates. If the included angle defined by two segments of a V-shaped tether is an acute angle (less than 90 degrees), lower flow shear is induced by the tether segments versus a V-shaped tether with an obtuse (greater than 90 degrees) included angle. Thus, in some embodiments, the venous valve prosthesis described herein includes a V-shaped tether with an acute angle relative to the wall of the anchoring member.

Another aspect of tether and ball interaction that discourages thrombus formation on the ball during antegrade venous flow is the provision of a tether that promotes dynamic movement of the ball in the open position. If a ball centered in the flow path exhibits small axial and lateral movement or flutter when the valve is open, this motion may prevent thrombus from forming on the surface of the ball. This motion is enabled by providing that a V-shaped tether is comprised of a material with suitably low bending stiffness. In addition, a linkage may be formed at the site of V-tether attachment to the ball, with the attachment of a small loop of either suture material or metal such as stainless steel to the ball, with secondary attachment of the V-tether to the loop on the ball. Similarly, the segments of the V-tether may attach to the eyelets, loops formed in the superelastic frame or directly to the membrane. These eyelets allow maximal tether limb mobility to enhance ball movement or flutter in the valve open position.

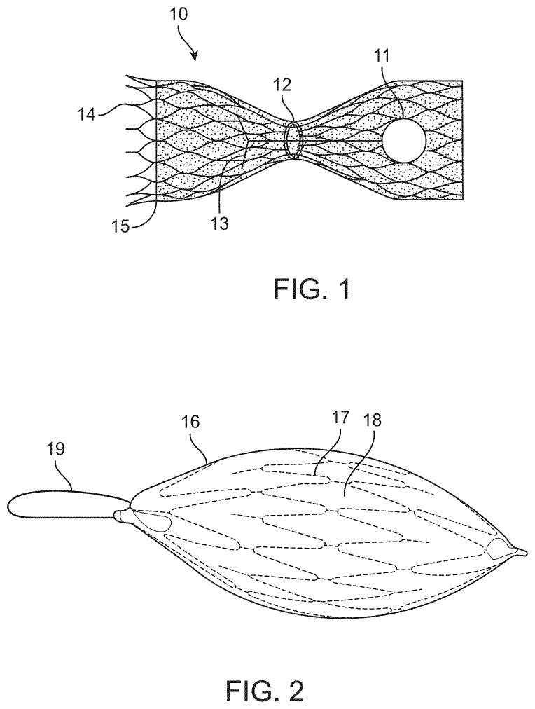

Referring now to FIG. 1, one example of a prosthetic venous valve 10 is illustrated in side view. In this embodiment, the valve 10 includes a self-expanding anchoring member 14 (or “frame”) that forms a lumen, a coating 15 (or “membrane”), a spherical ball 11, a valve seat 12, and a tether 13 that attaches to the ball 11 to the anchoring member 14. (In alternative embodiments, the ball may have any other suitable shape and the anchoring member may be completely or partially uncoated/uncovered.) In one embodiment, the anchoring member 14 is made of a self-expanding (e.g., superelastic) metal, such as but not limited to Nitinol. In alternative embodiments, the anchoring member 14 may be made of any type of high carbon steel, copper alloy, stainless steel, nickel alloy, alloy steel or composite (e.g., Ultem). The membrane 15 may be made of expanded polytetrafluoroethylene (ePTFE), in one embodiment, or alternatively any other suitable material. The membrane 15 may help direct fluid flow through the valve seat 12. The anchoring member 14 is self-expanding, and the proximal end and/or the distal end (or “apices”) of the anchoring member 14 on the proximal end and/or the distal end may protrude outward, into the wall of the vein, to secure the valve 10 in its implanted position. In some embodiments, anchoring protrusions (hooks, barbs, etc.) may be located somewhere between the proximal and distal ends of the anchoring member 14 and protrude radially outward from the surrounding lattice structure. The anchoring member 14 has an “hourglass” configuration, with a narrow central portion at the valve seat 12 and wide portions at its proximal and distal ends. The anchoring member 14 may be compressed into a smaller polymeric sheath or catheter for delivery to the implantation site. In some embodiments, the ball 11 is generally inelastic and cannot be compressed, thus limiting reduction of the delivery sheath size. In alternative embodiments, the ball 11 may be compressible and elastic, to allow for a smaller diameter delivery catheter.

FIG. 2 is a perspective view of another embodiment of a compressible ball 16 for use in a prosthetic venous valve, such as the valve 10 illustrated in FIG. 1. In this example, the ball 16 is made of a self-expanding (e.g., superelastic) metal lattice 17 covered by an ePTFE membrane 18. The compressible ball 16 has a prolate spheroid shape (i.e., football) shape, although in other embodiments it may have a round, ovoid teardrop or other suitable shape. A loop 19 composed of ePTFE or PTFE monofilament is attached to the compressible ball 16 at the site of the tether loop connection. The internal cavity of ball 16 may fill with blood (or related constituents) that may thrombose and organize into fibrous material after implantation. Alternatively, the cavity of the compressible ball 16 may be filled with open cell polymeric foam, such as polyurethane, to yield a solid structure following implantation.

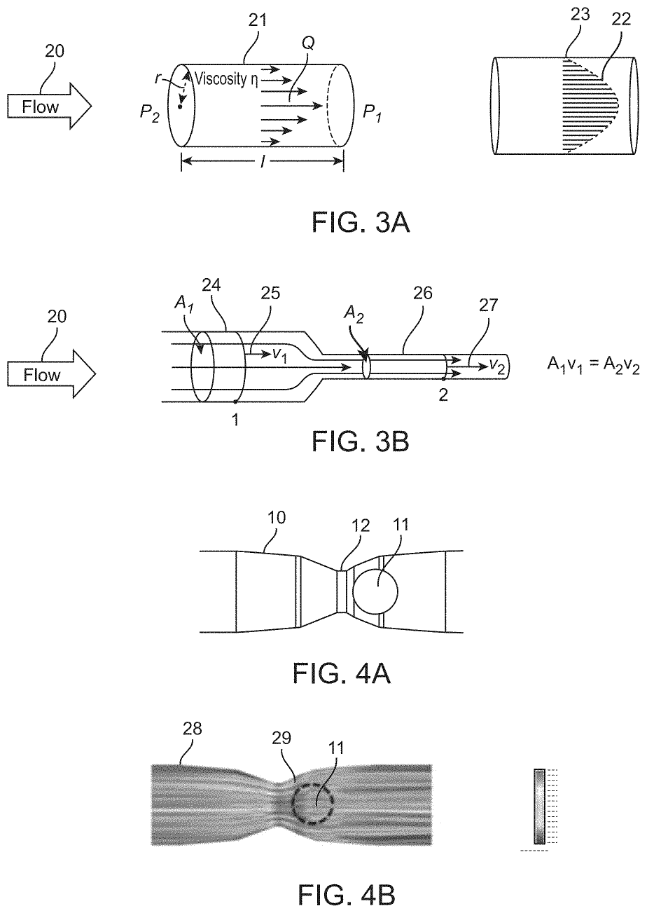

FIGS. 3a and 3b show the mechanics of blood flow in a vein and a tubular vein valve prosthesis. FIG. 3a depicts the parabolic velocity profile 22 of laminar blood flow 20 through the tubular structure of a vein 21. The velocity profile 22 follows Hagen-Poiseuille's law, which states that blood flow velocity is equal to zero at the inner wall 23 and increases to its maximum value in the center of the vein. FIG. 3b illustrates that with a constant fluid flow 20 of incompressible blood in a tubular structure, the following principle holds: A1v1=A2v2, where A1 is the cross-sectional area of the tube at point 1, A2 is the cross-sectional area of the tube at point 2, v1 is the velocity of blood at point 1, and v2 is the velocity of blood at point 2. A large diameter tube or vessel 24 will contain fluid flow 25 with less velocity than a smaller diameter tube 26, with corresponding higher velocity 27. Therefore, fluid velocity speeds up at areas of constriction in a vein or a venous valve implant.

FIG. 4a shows a prosthetic venous valve 10 in the open position, with the ball 11 displaced away from valve seat 12 due to antegrade flow (left to right in the illustration). FIG. 4b shows the corresponding flow velocity profile for the valve 10 in the open position. The highest and lowest velocities are shown as darker colors with the medium range velocities shown as lighter colors. The colors indicate that low flow velocity 28 exists as blood enters the valve 10, and the high velocity zone occurs as blood enters the reduced area valve seat 12 and flows past the ball 11.

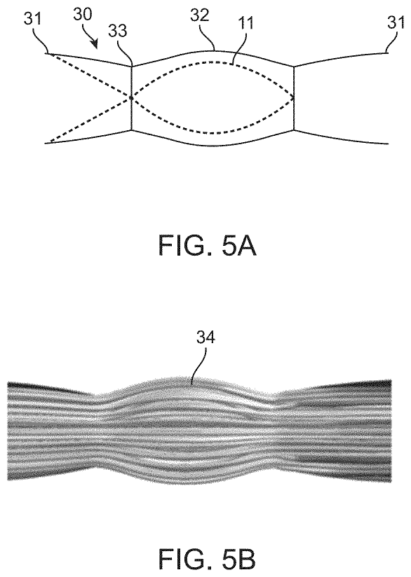

FIG. 5a shows the configuration of a constant flow velocity valve 30 that contains flared proximal and distal ends 31 to anchor to the vessel wall, and an expanded curved midsection 32 that is centered around the ball 11 in the open state. Due to the radius of curvature of the midsection 32 and the radius of the spherical ball 11, when the ball 11 is displaced from valve seat 33 in the constant flow velocity valve 30, the clearance open area between the ball 11 and the inner wall of valve 30 remains constant around the surface of ball 11. The resultant flow velocity profile 34 remains relatively uniform, as shown in FIG. 5b, by the absence of dark colored regions indicating high flow velocity streamlines. Regions of high velocity in the flow path of a valve may create eddy currents and areas of stagnant or recirculating flow that predispose to thrombus formation. Once thrombus is formed inside a valve, it may grow and propagate to cause valve and blood vessel occlusion. By maintaining a constant flow area around the ball 11, the flow characteristics of constant flow velocity valve 30 are optimized, and implant patency is likely improved. The configuration of the constant flow velocity valve 30, with proximal and distal flared ends 31 and curved midsection 32, may be described as having a “bowtie” shape, as opposed to the previously described “hourglass” shape.

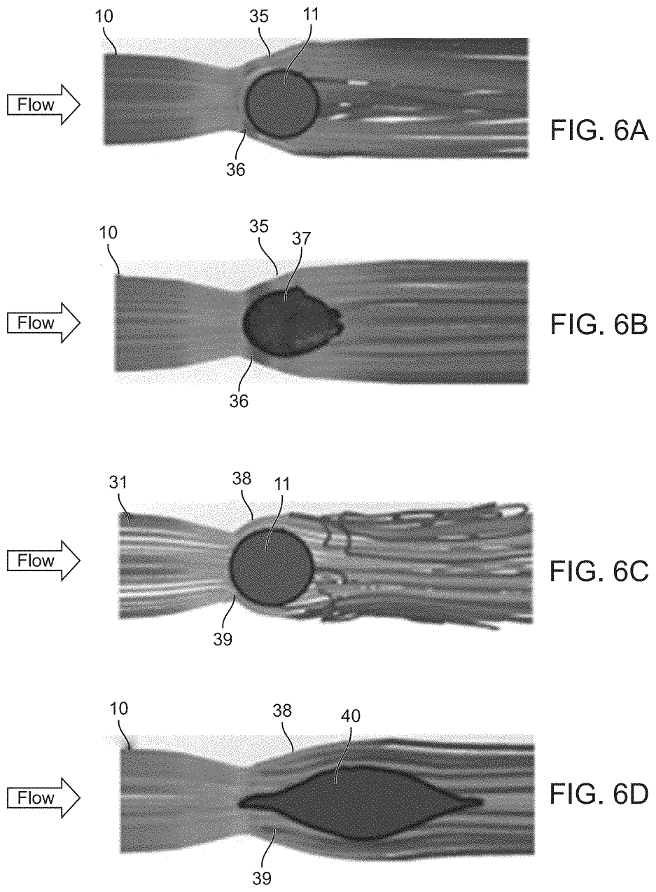

FIGS. 6a-6d illustrate flow patterns of a fluid (e.g., blood) through alternative examples of prosthetic venous valves. FIG. 6a shows valve 10 with a spherical ball 11, where the frame encompasses a straight section 35, as seen in an hourglass valve configuration. A dark vertical zone 36 in the flow path around ball 11 indicates the presence of high velocity flow. In FIG. 6b, an hourglass shaped valve 10 with straight frame section 35 is coupled with an egg-shaped ball 37. This combination results in a dark vertical zone 36 of high flow velocity. FIG. 6c depicts the flow velocity profile of a valve 31 configured with a curved frame section 38 and a spherical ball 11. The absence of a dark vertical zone 36 in the velocity profile 39 indicates uniform velocity flow around ball 11. This is the embodiment that maintains a constant area flow path around the inlet side of the ball. Darker low velocity streams are noted on the outlet side of the ball which indicate non uniform flow regains where the streamlines deviate laterally. FIG. 6d utilizes a frame 10 with a curved section 38, and a ball 40 in a prolate spheroid shape. The combination of a curved frame section 38 and a prolate spheroid ball 40 results in a uniform velocity profile 39 throughout the implant.

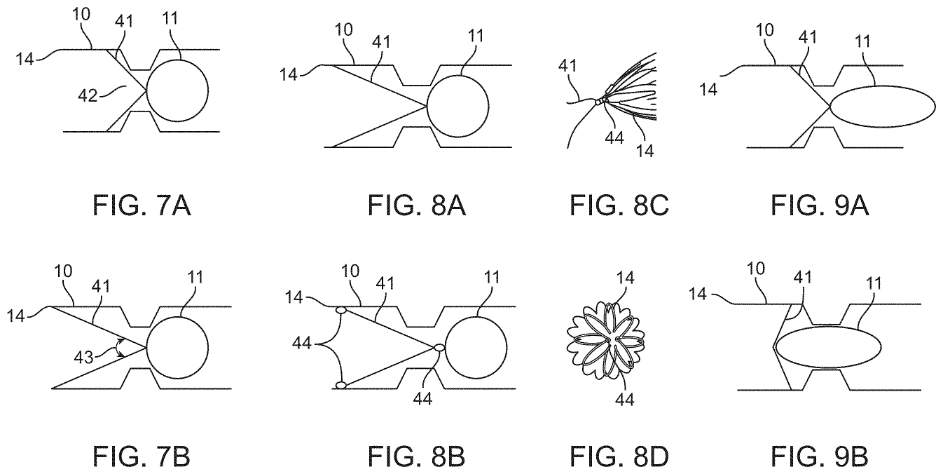

Referring now to FIGS. 7a and 7b, the geometry of the tether that attaches the ball to the frame also plays a role in the prevention (or formation) of thrombus within the valve. A V-shaped tether centers the ball within the valve to a greater degree than a single tether strand. FIG. 7a shows a valve 10 with ball 11 attached to the frame 14 (or “anchoring member”) via a V-shaped tether 41 that incorporates an obtuse angle 42 (greater than 90 degrees). The segments of the V-shaped tether 41 are short and attach to frame 14 in closer proximity to the ball 11. FIG. 7b shows a valve 10 with ball 11 attached to frame 14 via a V-shaped tether 41 that incorporates an acute angle 43 between its segments. In some embodiments, it may be advantageous to have the acute included angle 43 is be between 30 degrees and 70 degrees.

Provision of a small degree of ball motion when the valve is in the open position may prevent thrombus formation on the ball. FIG. 8a shows an attachment of ball 11 to valve 10 by means of a V-shaped tether 41 that is directly attached to the ball 11 and the frame 14. Increased axial ball movement and flutter (off axis motion) is achieved by the formation of loops 44 on the ball 11 and the frame 14 at the points of attachment of the V-shaped tether 41, as seen in FIG. 8b. The loops 44 may be formed of polymer monofilament material; or, in the case of frame 14, they may be formed integrally into the super elastic lattice of frame 14. V-shaped tether 41 may be formed of a single strand of monofilament of polymer, such as polytetrafluoroethylene (PTFE) or similar polymer, secured by tied square knots (or other suitable knot or constraint) to loops 44 on ball 11 and frame 14. Other forms of attachment include, but are not limited to, welding, adhesive, molding or the like. In one embodiment, the monofilament strand is flexible, to provide a higher degree of ball flutter and movement. FIG. 8c shows in close-up the attachment of the V-shaped tether 41 to loops 44 on the frame 14 of the prolate spheroid ball 40. FIG. 8d is an end view of the frame of prolate spheroid ball 40, showing that two loops 44 form the attachment point of V-shaped tether 41 to prolate spheroid ball 40.

Referring now to FIGS. 9a and 9b, an alternate embodiment of a prosthetic venous valve 10 is shown, in which an outer diameter of the ball 11 is close to an inner diameter of the orifice (or “valve seat”) of the frame 14. FIG. 9a shows the valve 10 in the open position, and FIG. 9b shows the valve 10 in the closed position, where the ball 11 resides fully within the valve seat orifice (thereby acting like a “plug”). This configuration may allow for a relatively small sized ball and/or a relatively large sized valve orifice, since in this embodiment the ball 11 is not oversized relative to the orifice. The V-shaped tether 41 may be formed of a single strand of monofilament of polymer, such as polytetrafluoroethylene (PTFE) or similar polymer, secured by square knots (or other suitable knot or constraint) to the ball 11 and frame 14. It may be configured as shown in FIGS. 9a and 9b, where the V-tethers 41 control both the open and closed positions of the valve, or alternatively inlet side V-tethers tethers may control the open position while outlet side V-tethers (attached to the opposite end of the ball) may independently control the closed position. Both the ball 11 and the orifice may contain portions that are cylindrically shaped to provide a seal zone when closed to reduce the need for critical positioning of the ball to achieve seal.

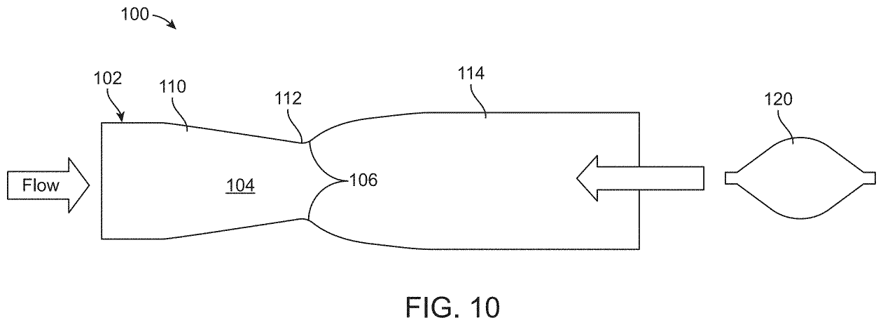

Referring now to FIG. 10, another embodiment of a venous valve prosthetic device 100 is illustrated in cross section. In this embodiment, the device 100 includes an expandable anchoring frame 102 and an expandable ball 120. The tether(s) are not shown in FIG. 10, although the device 100 would include at least one tether. The anchoring frame 102 includes an upstream portion 110, a middle portion 112 the inner surface of which forms a valve seat 106, and a downstream portion 114. In this embodiment, the shape of the anchoring frame 102 is asymmetrical, with the upstream portion 110 being shorter and more cone shaped or tapered, and the downstream portion 114 being longer and more cylindrical. This general shape may facilitate or enhance anchoring of and/or flow through the device 100. In general, the upstream portion 110 and the downstream portion 114 will expand to a larger diameter (or to different larger diameters) than the middle portion 112. In some embodiments, the middle portion 112 may expand sufficiently to still dilate the vein in which it is placed, although generally it will still be smaller when expanded than either the upstream portion 110 or the downstream portion 114. In this embodiment, the expandable ball 120 has a lemon shape. This shape may enhance the flow of blood around the ball 120.

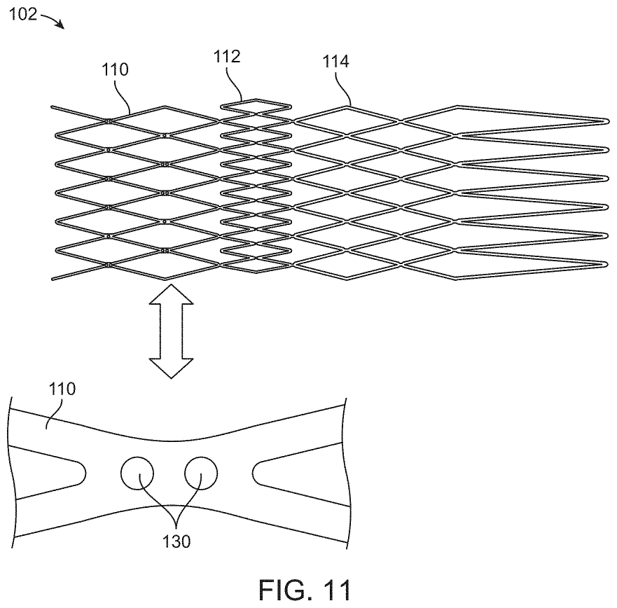

FIG. 11 illustrates one exemplary pattern for the lattice structure of the anchoring frame 102, with the frame 102 shown in a flattened configuration before complete assembly. The top panel of FIG. 11 shows the pattern for the upstream portion 110, the middle portion 112, and the downstream portion 114. As shown in the magnified bottom panel, the upstream portion 110 may include one or more apertures 130, through which one end of a tether and/or spring may be passed for attachment.

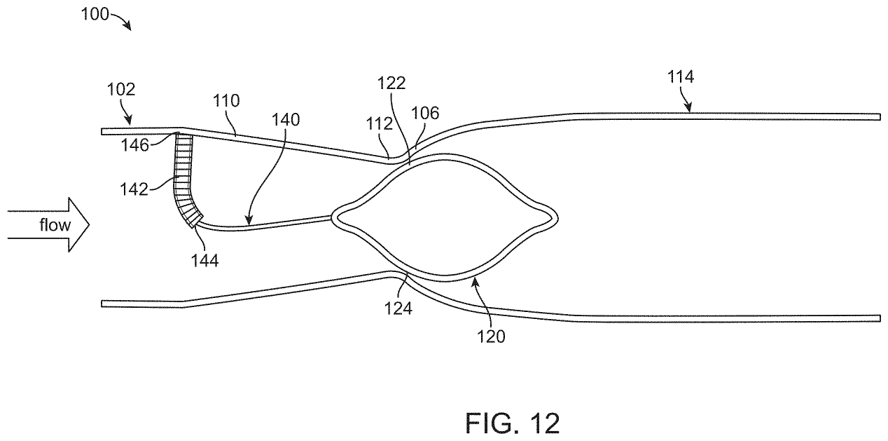

Referring now to FIG. 12, the same embodiment of the venous valve prosthetic device 100 is illustrated, with the addition of a tether 140 attached to a spring 142. In general, many of the embodiments described herein include a tether made of an elastic material, an elastic component used in conjunction with a tether, or both. This category of tethers and tether components may be referred to generally as “elastic tethers.” These tethers and components are designed to help improve flows properties and reduce shear and thrombogenesis risk, as detailed above. Various embodiments are designed to hold the tether out from the wall of the anchoring member and the vessel wall, and they also help pull the ball back into contact with the valve seat and allow for a desired amount of movement of the ball between the open and closed positions. Various embodiments of the elastic tethers and components are described further below.

In the embodiment of FIG. 12, one end of the ball 120 is attached to one end of the tether 140, such that two opposite sides 122, 124 of the ball either contact or come close to contacting the valve seat 106 of the anchoring frame 102 when the device 100 is in the closed position. (FIG. 12 illustrates the device in the nearly closed position, with a small gap between the sides 122, 124 and the valve seat, to allow for a small amount of blood flow when the valve prosthesis device 100 is subjected to low flow rates.) In this embodiment, the tether 140 is made of an inelastic or relatively inelastic material, such as a suture material, and it passes through an inner bore (or lumen) of the spring 142 to attach to the upstream portion 110 of the anchoring member 102. The spring 142 has a proximal end 146 that attaches to the anchoring member 102 and a distal open end 144. Although the tether 140 itself is not elastic (or not significantly elastic), the spring 142 confers some measure of elasticity to the function of the tether 140 by flexing in the horizontal (i.e., upstream/downstream) orientation. The spring 142 also beneficially holds the tether 140 out from the inner wall of the anchoring member 102, thus preventing the tether 140 from resting against the inner wall when in a slack configuration. This may help prevent clot formation within the device 100.

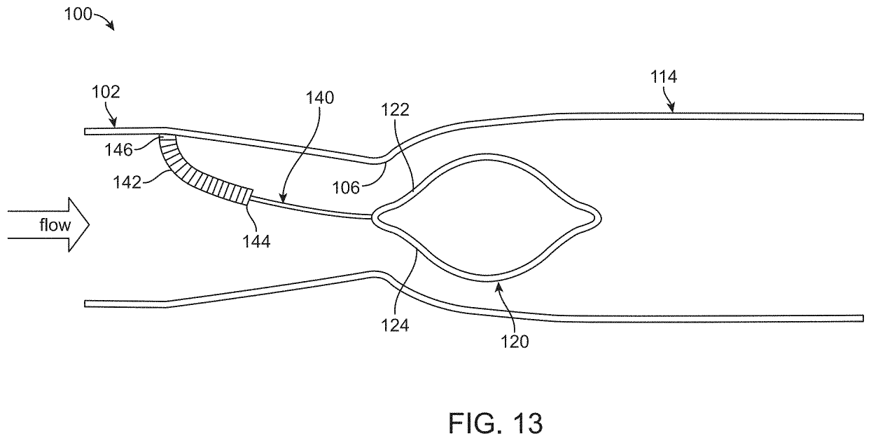

FIG. 13 shows the same venous valve prosthetic device 100 in the open position, when blood is more actively flowing through the device 100. This figure illustrates the generally horizontal flexing of the spring 142. When flow decreases, the spring 142 naturally moves back to a more vertical position, as in FIG. 12, thus pulling the tether 140 and the ball 120 into a more closed position.

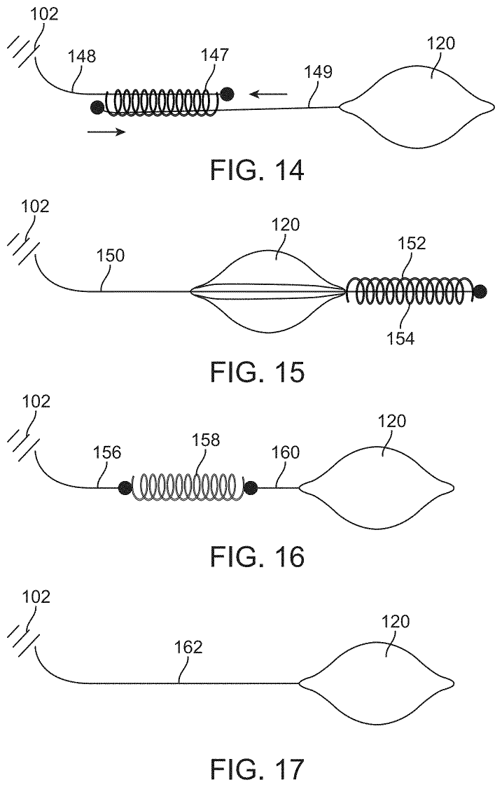

Referring now to FIGS. 14-16, four alternative embodiments for tethering the ball 120 to the anchoring frame 102 are illustrated. Each example includes some mechanism for providing elasticity to the tether. For example, in FIG. 14, the embodiment includes a first tether portion 148 and an overlapping second tether portion 149, with a spring 147 disposed over the overlapping ends of the two tether portions 148, 149. The embodiment of FIG. 15 includes a tether 150 that passes through the ball 120 and has a spring 152 positioned over a distal portion of it. FIG. 16 shows an embodiment where a first tether portion 156 is attached to a second tether portion 160 via a spring 158 residing between the two. Finally, in FIG. 17, there is no spring component, but the tether 162 itself is made of an elastic material. Examples of elastic materials the tether 162 may be made of include, but are not limited to, neoprene, silicone, ethylene propylene diene monomer (EPDM), nitrile, buna-N, styrene-butadiene rubber (SBR) and rubber elastomers. In any of the embodiments described herein, a tether may be made of an elastic material, even if a spring or other elastic component is also included. In any given embodiment, any of the features of these various embodiments may be combined.

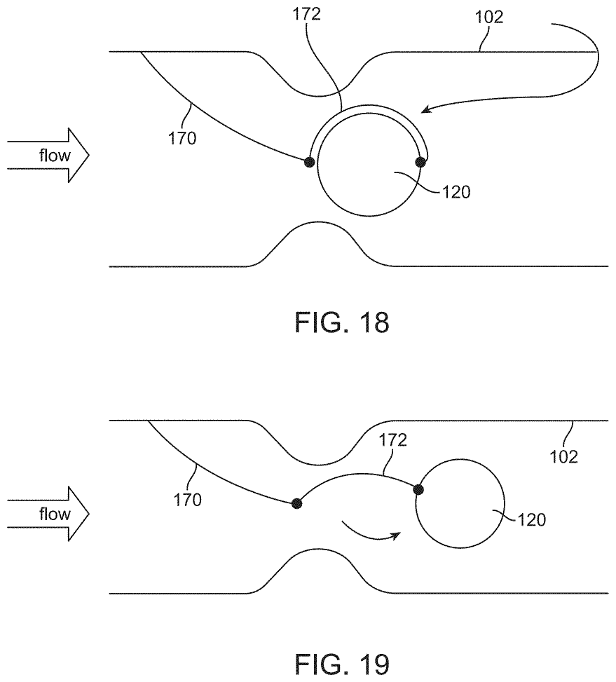

FIGS. 18-19 illustrate another embodiment of a tether 170 for a venous valve prosthetic device 100. In this embodiment, the tether 170 is attached to a distal extension curved beam spring 172, which in turn is attached to the ball 120. In the closed position, as in FIG. 18, the ball 120 resides within the distal extension curved beam spring 172. In the open position, as in FIG. 19, the ball 120 flips out of the distal extension curved beam spring 172 and the curved beam spring flexes to open the valve but remains attached to the distal end of the distal extension curved beam spring 172. When flow is reduced, the ball 120 flips back into the distal extension curved beam spring as in FIG. 18.

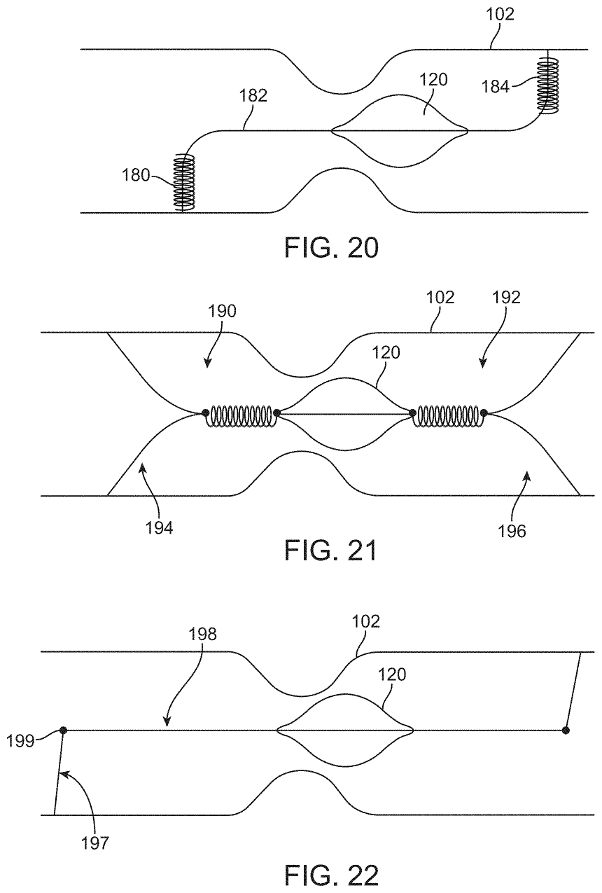

Referring now to FIGS. 20-22, three alternative embodiments of tethering systems that include tether attachments on both the upstream portion 110 and the downstream portion 114 are illustrated. In FIG. 20, the embodiment includes a tether 182, a first spring 180 attached to the upstream portion 110 of the anchoring member 102, and a second spring 184 attached to the downstream portion 114. The two springs 180, 184 help hold the ball 120 and the tether 182 out from the inner wall of the anchoring member 102 and also provide horizontal flexing.

FIG. 21 shows an embodiment with a first V-shaped tether 194, a first spring 190, a second V-shaped tether 196, and a second spring 192. Each of the two V-shaped tethers 194, 196 attaches to the anchoring member 102 at two attachment points. In the embodiment of FIG. 22, each of the two tethering systems includes a first beam spring 197 and a first tether portion 198, attached together at a connection 199. Again, any suitable combination of any of the features or components shown in FIGS. 20-22 may be made in an alternative embodiment.

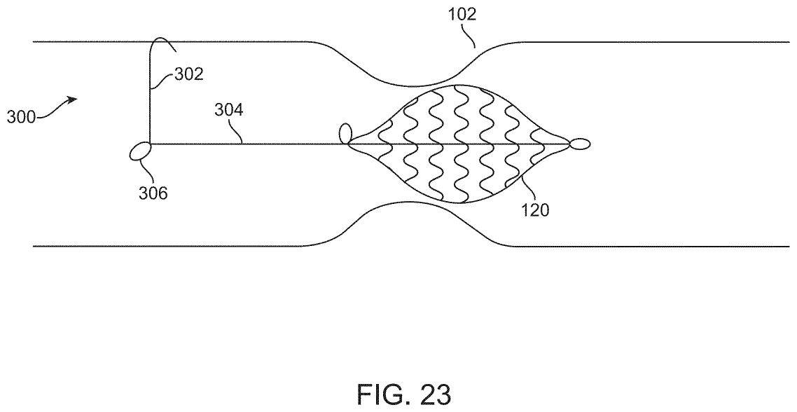

FIG. 23 illustrates yet another tethering embodiment. In this embodiment, the tether spring system 300 includes a first portion 302 and a second portion 304, attached at a hinge 306. The first portion 302 holds the rest of the spring system 300 out from the inner wall of the anchoring member. The torsion spring 306 allows for flexing of the spring system 300 in the horizontal direction.

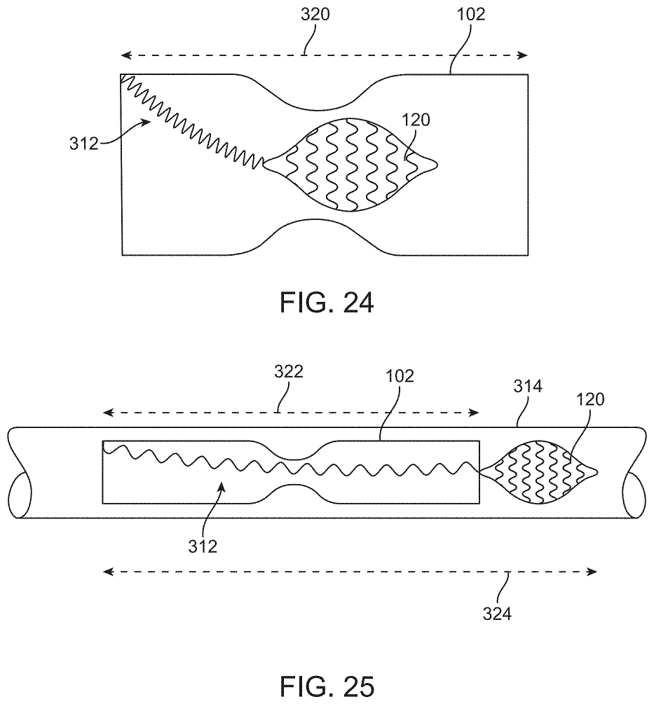

Referring now to FIGS. 24 and 25, another embodiment of venous valve prosthetic device 100 is illustrated. FIG. 24 shows the device 100 in its expanded (or “default”) configuration, as it would appear when deployed in a vein. It is also shown in the valve-closed position, with the ball 120 residing in the valve seat 106. In this embodiment, the tether 312 is a spring, and the valve prosthesis device 100 has a length 320 in the default configuration. FIG. 25 shows the valve device 100 in a stretched, collapsed position, inside of a catheter 314 for delivery into the vein (i.e., before deployment). In this embodiment, the spring tether 312 allows the ball 120 to reside outside of the anchoring member 102 but within the catheter 314 during delivery, thus allowing the overall diameter of the venous valve prosthetic device 100 to be smaller (since the ball 120 doesn't need to reside within the anchoring member 102 during delivery). In this delivery configuration, the anchoring member 102 has a length 322, and the anchoring member 102 plus the ball 120 have a length 324, both of which are longer than the length 320 of the device 100 in its expanded configuration.

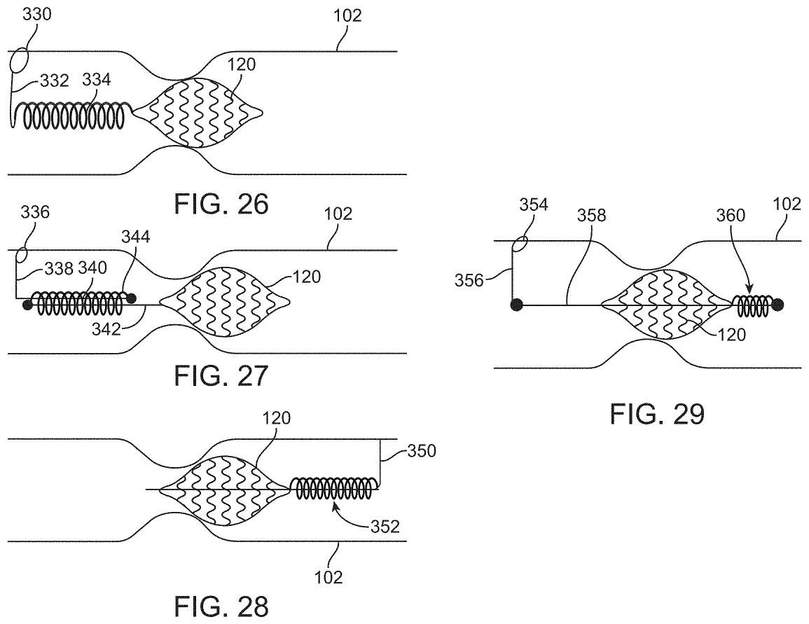

Referring now to FIGS. 26-29, additional alternative embodiments for ball tethering systems are illustrated. FIG. 26 shows an embodiment in which a first tether portion 332 is attached via an attachment point 330 to the anchoring frame 102 and via an opposite end to a second tether portion 334 that is formed as a spring. In the embodiment of FIG. 27, a first tether portion 338 is attached via an attachment point 336 to the anchoring frame 102. A second tether portion 342 is attached to the ball 120. Overlapping portions 340 of both tether portions 338. 342 are attached to one another via a spring 344. The embodiment of FIG. 28 includes a tether 350 attached to the downstream portion of the anchoring member 102 and extending through a spring 352 and the ball 120. Finally, in FIG. 29, the embodiment includes a first tether portion 356 attached to the anchoring frame 102 at an attachment point 354, and a second tether portion 358 that extends through the ball 120 and through a distal spring 360. Again, any of these features or components may be combined to form an alternative embodiment.

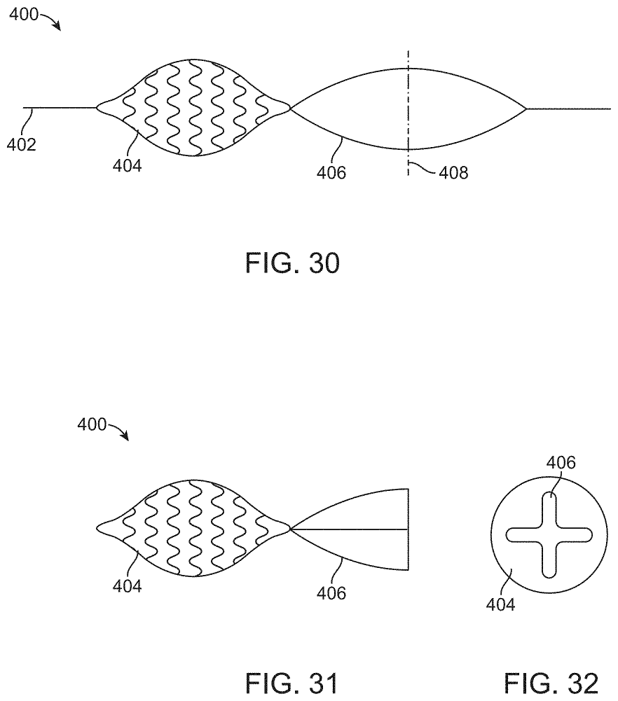

Referring now to FIGS. 30-32, in some embodiments it might be beneficial to have a ball 400 for a venous valve implant, with a fin 406 to enhance blood flow around the ball. FIG. 30 shows the ball 400 during manufacturing, with a lattice structure 404, an illustrative tether portion 402, the fin 406, and a cut line 408 showing where the fin 406 can be cut during manufacturing. FIG. 31 is a side view of the ball 400 with the fin 406, when complete. FIG. 32 is a posterior view of the ball 400 with the fin 406.

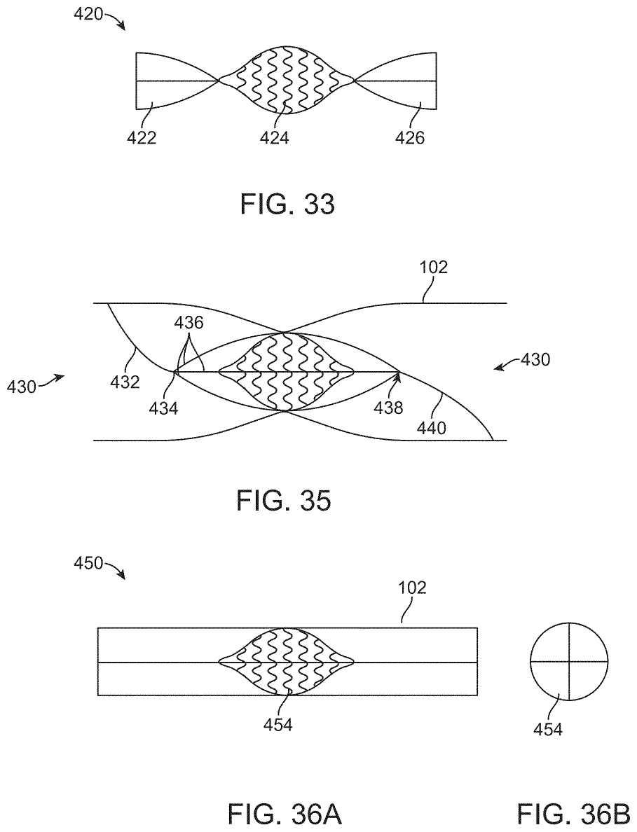

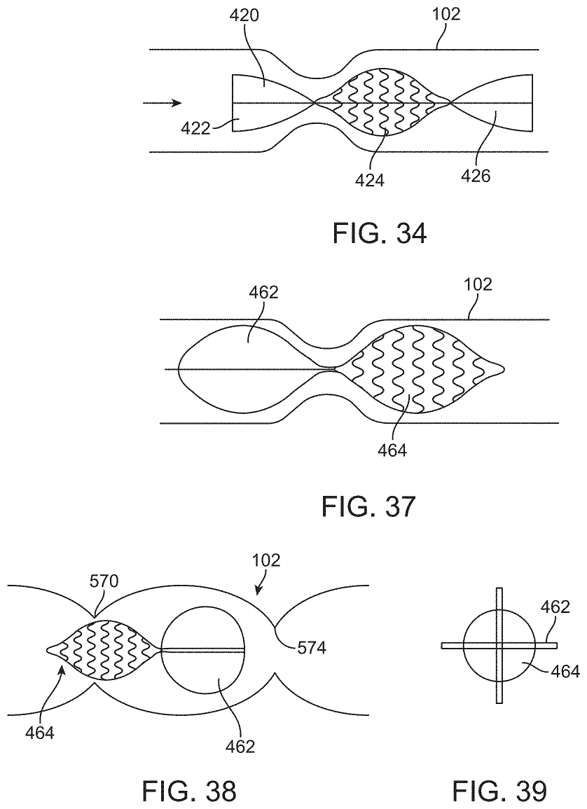

FIGS. 33 and 34 are side views of an alternative embodiment of an expandable ball 420 for a venous valve implant. In this embodiment, the ball 420 includes a central ball portion 424 and two fins 422, 426 on either side of the ball portion 424.

FIG. 35 is a side view of another embodiment of an expandable ball 430, attached via two tethers 432, 440 to the anchoring frame 102. The ball 430 includes two fins 436, which are attached at attachment points 434, 438 to the two tethers 432, 440.

FIGS. 36A and 36B are side and posterior views, respectively, of an alternative embodiment of an expandable ball 450. In this embodiment, the expandable ball 450 has a plug shape 454 with long rectangular fins.

FIG. 37 illustrates another embodiment of an expandable ball. In this embodiment, the expandable ball has a figure-eight shape and includes a first portion 462 that expands in the upstream portion 110 of the anchoring frame 102 and a second portion 464 that expands in the downstream portion 114. The ball thus tethers itself, due to its size and shape, and will move back and forth relative to the valve seat without exiting the device 100.

FIGS. 38 and 39 are side and posterior views, respectively, of another alternative embodiment of an expandable ball 464 with a fin 462. In this embodiment, the anchoring frame includes a first valve seat 570 and a second valve seat 574, and the ball 464 is trapped between the two so cannot exit the device 100.

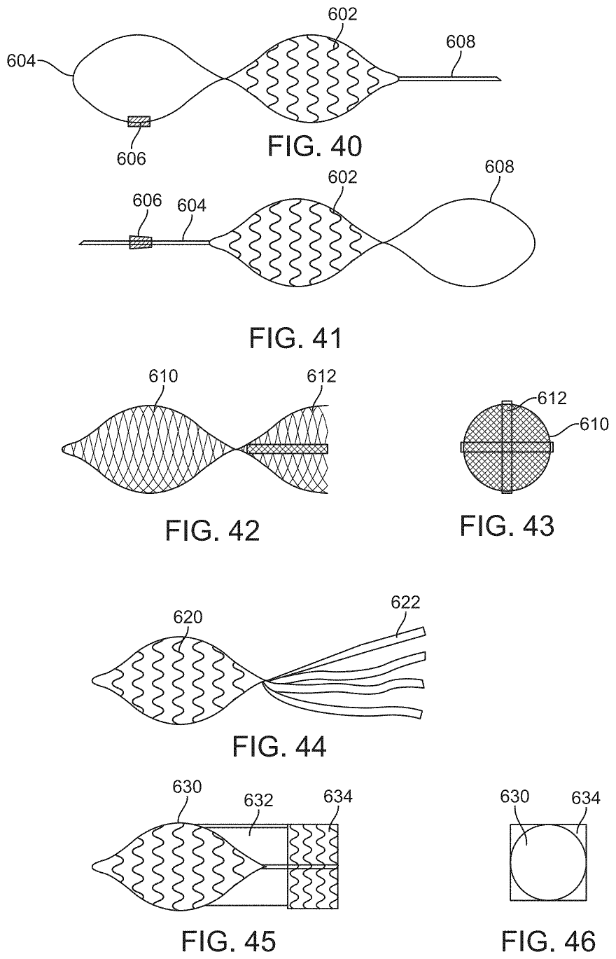

FIGS. 40 and 41 are side views of yet another embodiment of an expandable ball 602 for use in a venous valve prosthetic device 100. In this embodiment, the ball 602 is coupled with first fin 604 (or “hemofoil”) and second fin 608. The first fin 604 also includes a radiopaque marker 606. The two fins 604, 608 are oriented at ninety degree angles from one another. This is evident from the figures, as the ball 602 and fins 604, 608 are rotated ninety degrees in FIG. 41, relative to FIG. 40.

FIGS. 42 and 43 are side and posterior views, respectively, of another alternative embodiment of a ball 610 with a hemofoil 612 (or “fin”). In this embodiment, the ball 610 and the hemofoil 612 are made of a braided material, such as a braided wire mesh.

FIG. 44 is a side view of another example of an expandable ball 620, with this embodiment including a fin 622 made of multiple strips of material. One example of such material is polytetrafluoroethylene (PTFE), although other materials can be used. The fin 622 of material strips may increase drag force to promote lift of the ball 620.

FIGS. 45 and 46 are side and posterior views, respectively, of yet another example of an expandable ball 630. In this embodiment, the ball 630 is connected to multiple strips of material 632, which are in turn connected to a box-like structure 634 to promote drag. Overall, this structure is similar to that of a box kite.

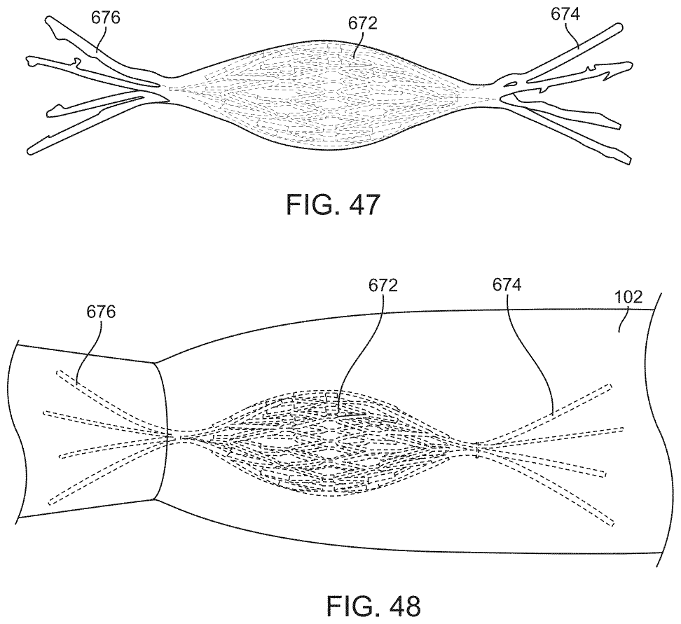

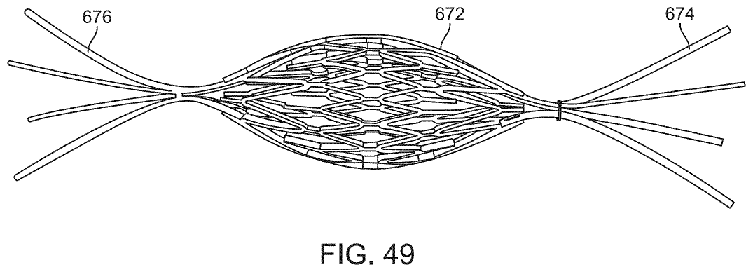

Referring now to FIGS. 47-49, three side views of the same expandable ball 672 are illustrated. The ball 672 is shown coated with PTFE in FIG. 47, placed within an artificial test vessel in FIG. 48, and uncoated in FIG. 49. In this embodiment, the expandable ball 672 includes multiple proximal fingers 676 and multiple distal fingers 674. The fingers 674, 676 help center the ball 672 within the venous valve prosthetic device and also may have tethering effects. In various embodiments, the ball 672 and the fingers 674, 676 may be coated (or covered) or uncoated. When the ball 672 is covered, the cover (or coating) may include one or more holes or apertures to allow blood to enter and fill the ball 672. This may help the ball 672 achieve a desired weight and/or buoyancy in blood. These holes or apertures in a coating or covering of the ball 672 may be included in any of the embodiments described in this disclosure.

The foregoing is believed to be a complete and accurate description of the invention. In alternative embodiments, however, any of the described features may combined in different ways or altered, without departing from the scope of the invention.

1. A prosthetic venous valve, comprising:
an expanding anchoring frame having an upstream end, a downstream end, a middle portion and a lumen extending through the anchoring frame from the upstream end to the downstream end;
a valve seat comprising a portion of the middle portion of the anchoring frame;
a ball disposed within the lumen of the anchoring frame, wherein the ball moves between an open position, in which the ball is located apart from the valve seat, and a closed position, in which the ball is located in contact with or near the valve seat to reduce or prevent backflow of blood through the prosthetic venous valve; and
at least one ball retention tether coupled with the ball and the anchoring frame, wherein the at least one ball retention tether comprises at least one elastic component or material.

2. The prosthetic venous valve of claim 1, further comprising a membrane disposed over at least part of the anchoring frame, wherein the membrane is made of at least one substance selected from the group consisting of polymers, hyaluronic acid, heparin and anticoagulant agents.

3. The prosthetic venous valve of claim 1, wherein the anchoring frame has an asymmetric shape, and wherein a downstream portion of the anchoring frame is longer than an upstream portion of the anchoring frame.

4. The prosthetic venous valve of claim 1, wherein the valve seat comprises a tapered portion of the middle portion of the anchoring frame.

5. The prosthetic venous valve of claim 1, wherein the anchoring frame has a shape selected from the group consisting of an hourglass shape, a bowtie shape and an asymmetrical shape.

6. The prosthetic venous valve of claim 1, wherein an inner diameter of the valve seat is larger than a largest outer diameter of the ball by 0.5 millimeters or less, thus allowing the ball to pass into the valve seat and act as a plug.

7. The prosthetic venous valve of claim 1, wherein the ball has a shape selected from the group comprising spherical, prolate spheroid, ellipsoid, ovoid, egg shaped, lemon shaped and asymmetrical.

8. The prosthetic venous valve of claim 1, wherein the ball is expandable and comprises:
an expandable lattice; and
a membrane covering the expandable lattice.

9. The prosthetic venous valve of claim 8, wherein the membrane includes at least one aperture for allowing blood to pass into an interior of the ball.

10. The prosthetic venous valve of claim 1, wherein the at least one ball retention tether comprises a V-shape with a first end attached to the anchoring frame, a middle portion attached to the ball, and a second end attached to the anchoring frame.

11. The prosthetic venous valve of claim 1, wherein the at least one ball retention tether comprises a main tether member, attached at one end to the ball and at an opposite end to the anchoring frame, and wherein the at least one elastic component comprises a spring disposed over a portion of the main tether member and attached at one end to the anchoring frame.

12. The prosthetic venous valve of claim 11, wherein the main tether member is attached to eyelets on the ball and on the anchoring frame.

13. The prosthetic venous valve of claim 11, wherein the main tether member is attached to a covering on the ball and to a covering on the anchoring frame.

14. The prosthetic venous valve of claim 1, wherein the at least one ball retention tether comprises:
a first tether attached to the anchoring frame; and
a second tether attached to the ball,
wherein the at least one elastic member comprises a spring connecting the first tether to the second tether.

15. The prosthetic venous valve of claim 1, wherein the at least one ball retention tether is made of an elastic material.

16. The prosthetic venous valve of claim 1, wherein the at least one ball retention tether comprises:
an upstream tether attaching the ball to an upstream portion of the anchoring member; and
a downstream tether attaching the ball to a downstream portion of the anchoring member.

17. The prosthetic venous valve of claim 1, wherein the at least one ball retention tether comprises:
multiple upstream expandable fingers extending from an upstream end of the ball; and
multiple downstream expandable fingers extending from a downstream end of the ball.

18. The prosthetic venous valve of claim 1, wherein the at least one ball retention tether comprises:
a first tether attached to the anchoring frame; and
a second tether attached to the ball,
wherein the at least one elastic member comprises a hinge connecting the first tether to the second tether.

19. The prosthetic venous valve of claim 1, wherein the ball further comprises a loop for connecting the at least one ball retention tether to the ball.

20. A prosthetic venous valve, comprising:
an expanding tubular anchoring frame extending from a first end to a second end of the venous valve prosthesis, forming a lumen;
a valve seat formed by or attached to the anchoring frame;
a ball in the lumen of the anchoring frame; and
at least one ball retention tether coupled to the ball and the anchoring frame, wherein the at least one ball retention tether comprises at least one elastic component or material.

21. The prosthetic venous valve of claim 20, wherein the at least one elastic component comprises a spring disposed over a main tether member and attached to the anchoring frame.

22. The prosthetic venous valve of claim 20, wherein the ball and the at least one ball retention tether are configured so that the ball moves between an open position, in which the ball is located downstream of the valve seat, and a closed position, in which the ball is located closer to the valve seat or contacts the valve seat, to reduce or prevent backflow of blood.

23. The prosthetic venous valve of claim 20, wherein the ball is expandable from a compressed configuration for delivery into the vein through a catheter to an expanded configuration outside the catheter.
